(19) United States
(12) Patent Application Publication
(10) Pub. No.: US 2008/0300587 A1
(43) Pub. Date: Dec. 4, 2008

(54) Heat Treatment Catheter

(75) Inventor: Neil L. Anderson, Roseville (AU)

Correspondence Address: MORRISON & FOERSTER LLP, 755 PAGE MILL RD, PALO ALTO, CA, 94304-1018, US

(73) Assignee: CATHRX LTD

(21) Appl. No.: 11/885,029

(22) PCT Filed: Feb. 23, 2006
(86) PCT No.: PCT/AU2006/000227

§ 371 Date: Jul. 29, 2008

Related U.S. Application Data
(60) Provisional application No. 60/658,246, filed on Mar. 2, 2005.

(51) Int. Cl. A61B 18/08

(52) U.S. Cl. 606/29

(57) ABSTRACT

A heat treatment catheter (10) for use in heating a biological site in a patient's body includes an elongate electrode-carrying element (12). A plurality of heating electrodes (16) are arranged at spaced intervals at a distal region (14) of the electrode-carrying element (12). The distal region (14) of the electrode-carrying element (12) is formed into a predetermined, non-rectilinear shape so that, in use, when any two of the electrodes (16) are energised with heat energy, overlapping heat treated zones (36, 38) are created by the energised electrodes (16) to form a heat treated region extending between the two energised electrodes (16).

### CROSS-REFERENCE TO RELATED APPLICATIONS

The present application claims priority from United States of America Provisional Patent Application No. 60/658,246 filed on 2 Mar. 2005, the contents of which are incorporated herein by reference.

### FIELD OF THE INVENTION

This invention relates, generally, to a heat treatment catheter and, more particularly, to a system for, and a method of, heating a biological site in a patient's body.

### BACKGROUND OF THE INVENTION

Heat treatments such as ablative techniques for forming lesions at a biological site in a patient's body, for example, for the treatment of heart arrhythmias, are becoming increasingly prevalent. Conventionally, electrodes are energised with radio frequency energy to effect ablation at the biological site. To form a longer or larger lesion at the site generally involves forming a first lesion using the electrode in an initial position and then re-positioning the electrode, at least one more time, relative to the first lesion to increase the length or size of the lesion. Other heat treatments such as, for example, in the treatment of Parkinsons disease, tumour ablation, endometriosis and pain management, are also being increasingly used.

It will be appreciated that such manoeuvring of the electrode needs to be done by a clinician using a fluoroscope or similar device and involves very careful placement of the electrode. Further, all the RF energy is concentrated in the one electrode resulting in a deeper lesion being formed than may be necessary. This can have adverse consequences for the patient.

### SUMMARY OF THE INVENTION

According to a first aspect of the invention, there is provided a heat treatment catheter for use in heating a biological site in a patient's body, the heat treatment catheter including:
an elongate electrode-carrying element;
a plurality of heating electrodes arranged at spaced intervals at a distal region of the electrode-carrying element; and
the distal region of the electrode-carrying element being formed into a predetermined, non-rectilinear shape so that, in use, when any two of the electrodes are energised with heat energy, overlapping heat treated zones are created by the energised electrodes to form a heat treated region extending between the two energised electrodes.

By “non-rectilinear” is meant that, in a rest configuration, the distal region of the electrode-carrying element forms a shape other than a straight, non-curved shape.

The predetermined shape of the electrode-carrying element may be a coiled or spiral shape. The coiled or spiral shape of the distal region of the electrode-carrying element may lie in a plane substantially transverse to a longitudinal axis of a remainder of the electrode-carrying element. The electrode-carrying element may be sufficiently flexible so that, at least when the spiral shape distal region of the electrode-carrying element is urged against the site to be treated, the spiral shape is able mould to the shape of the site and flex to accommodate surface irregularities at the site. It will be appreciated that, generally, in the treatment of arrhythmias, tissue of a heart wall of the heart has surface irregularities which need to be taken into consideration for obtaining suitable electrode-tissue contact. With the provision of a flexible distal region of the electrode-carrying element, the surface irregularities can, at least to a certain extent, be accommodated.

Preferably, the electrode-carrying element is steerable. Thus, the electrode-carrying element may have a lumen in which a steering mechanism is received.

The electrode-carrying element may, in a preferred embodiment, be manufactured in accordance with the Applicant's manufacturing technique as described in the Applicant's International Patent Application No. PCT/AU01/01339 entitled “An electrical lead” dated 19 Oct. 2001. The contents of that International Application are incorporated in this specification by reference.

Preferably, an electrode at a distal end of the electrode carrying element and any one other electrode are energised simultaneously with the heat energy, which may be ablating energy. In addition, the electrodes to be energised simultaneously may be energised by out-of-phase electrical sources. These out-of-phase sources may be provided by means of a transformer as described in the Applicant's International Application No. PCT/AU2003/001421 entitled “System for, and method of, heating a biological site in a patient's body and dated 28 Oct. 2003 (International Publication No. WO 2004/039274). Once again, the contents of that International Application are incorporated in this specification by reference.

Hence, according to a second aspect of the invention, there is provided a system for heating a biological site in a patient's body, the system including:
an electrode-carrying element having a plurality of electrodes arranged at spaced intervals at a distal region, the distal region of the electrode-carrying element being arranged in a predetermined non-rectilinear shape; and
a source of electromagnetic energy connectable to the electrode-carrying element to energise at least two of the electrodes simultaneously, with the electromagnetic energy associated with one electrode being out of phase with the electromagnetic energy associated with any one other, simultaneously energised electrode.

The source of electromagnetic energy may be a transformer having a primary winding and a secondary winding, the secondary winding having at least one tap to provide a ground reference and at least two sources of heat energy such as, for example, radio-frequency (RF) energy.

A secondary winding of the transformer may have a 1:1 ratio with respect to a primary winding of the transformer.

Preferably, the electrode at a distal end of the electrode-carrying element is always connected to one of the sources of heat energy. The system may include a switching arrangement connectable to the other source of heat energy for switching any one of the remaining electrodes, at any one time, into electrical contact with that other source of heat energy.

The primary winding of the transformer may be connectable to an energy generator for supplying the heat energy to the primary winding of the transformer.

The secondary windings of the transformer may supply energy to the electrodes connected to the secondary windings with the energy supplied to one of the electrodes being 180° out of phase with the energy supplied to the other connected electrode.

According to a third aspect of the invention, there is provided a method of heating a site in a patient's body, the method including:
positioning a distal region of an electrode-carrying element relative to the site, the distal region of the electrode-carrying element having a plurality of electrodes arranged at spaced intervals and the distal region of the electrode-carrying element being non-rectilinear in shape;
supplying energy to at least two of the electrodes simultaneously with the energy supplied to each of the electrodes being out of phase; and
maintaining the energy supply to the energised electrodes until overlapping heat treated zones are formed by the electrodes to form a heat treated region extending between the two energised electrodes.

As indicated above, the distal region of the electrode-carrying element may be in the form of a spiral or coiled shape so that the electrodes lie in spaced relationship on adjacent, but spaced, turns of the spiral shaped end of the electrode-carrying element. Thus, the method may include forming overlapping zones by using the electrode at the distal end of the electrode-carrying element and at least one other electrode to form the desired shape of heat treated zone, which, in the case of ablation techniques, may be a lesion, in the desired position at the site.

### BRIEF DESCRIPTION OF THE DRAWINGS

FIG. 1 shows a schematic, three dimensional view of part of a heat treatment catheter, in accordance with an embodiment of the invention;

FIG. 2 shows a cross-sectional view of the catheter taken along line II-II in FIG. 1 and

FIG. 3 shows a schematic block diagram of a system, also in accordance with an embodiment of the invention, for heating a site in a patient's body.

### DESCRIPTION OF EXEMPLARY EMBODIMENT

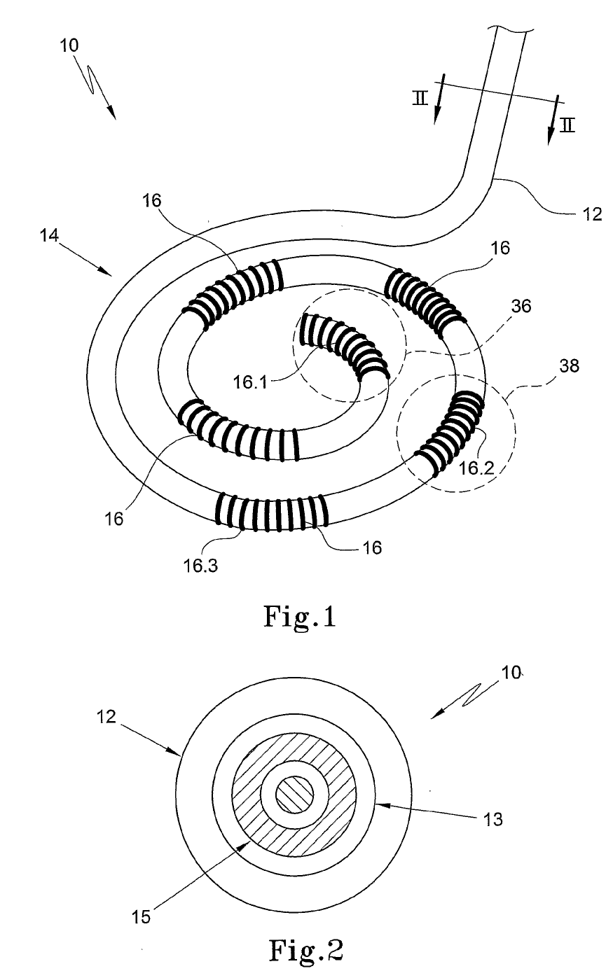

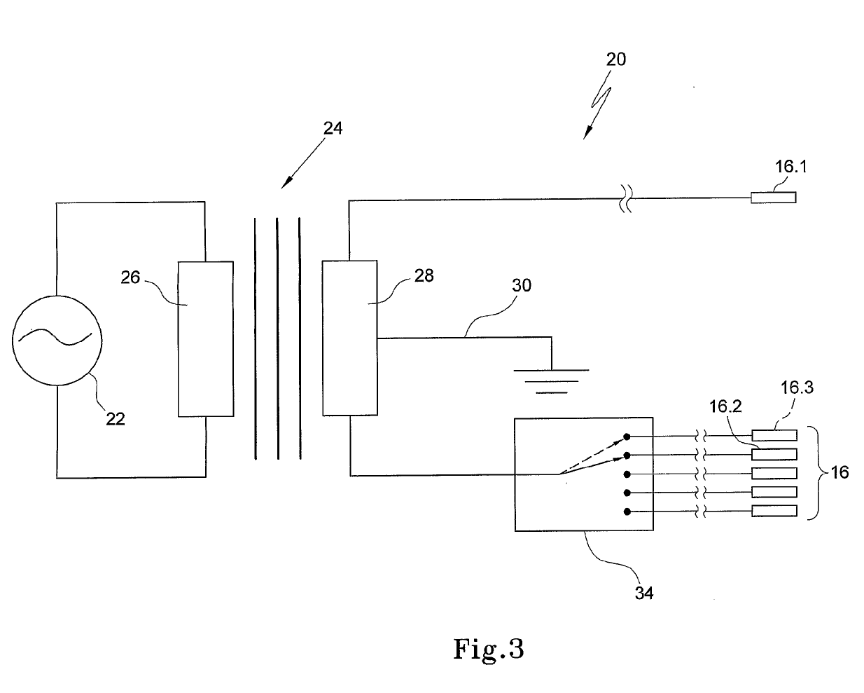

Referring initially to FIG. 1 of the drawings, reference numeral 10 generally designates a heat treatment catheter in accordance with an embodiment of the invention. The catheter 10 will be described below with reference to its application in ablation. It will, however, be appreciated that the catheter 10 could equally be used in other heat treatment applications, such as pain management, by appropriately controlling the intensity of the energy emitted by electrodes of the catheter 10.

The catheter 10 includes an elongate electrode-carrying element or electrode sheath 12. A distal region 14 of the electrode sheath 12 is pre-formed into a predetermined non-rectilinear shape. The formation of the non-rectilinear shape at the distal region 14 of the electrode sheath 12 may be effected by way of a stylet 15 received in a lumen 13 of the electrode sheath 12. The stylet 15 may, conveniently, also be a steerable stylet, or steering shaft, to steer the distal region 14 of the electrode sheath 12. An example of a steering shaft 15 that can be used is described in the Applicant's co-pending International Application No. PCT/AU2005/000216 entitled “A steerable catheter” dated Feb. 18, 2005, the contents of which are incorporated in this specification by reference.

As indicated above, the electrode sheath 12 is, conveniently, fabricated in accordance with the Applicant's fabrication technique as described in its International Application No. PCT/AU01/01339. This provides an unimpeded lumen 13 through which the steering shaft 15 is inserted.

In the illustrated embodiment, the distal region 14 of the electrode sheath 12 of the catheter 10 is formed into a spiral shape. Conveniently, the spiral shape of the distal region 14 of the electrode sheath is imparted by the steering shaft 15 which is formed of shape memory alloy, such as nitinol, and is pre-formed into the desired shape. The spiral shaped distal region 14 lies in a plane substantially transverse to a longitudinal axis of the remainder of the electrode sheath 12.

A plurality of heating, or ablation, electrodes 16 are carried on the distal region 14 of the electrode sheath 12. One of the electrodes 16.1 is an end electrode arranged at a free end of the spiral.

The spiral configuration of the distal region 14 of the electrode sheath 12 ensures that electrodes are arranged in spaced, adjacent relationship on adjacent, spaced turns of the spiral.

In FIG. 2 of the drawings a system, in accordance with another embodiment of the invention, for heating a site in a patient's body is illustrated and is designated generally by the reference numeral 20. Once again the system 20 is described with reference to its application in ablating at the site. The system 20 includes a generator 22 for generating electromagnetic energy in the form of radio frequency (RF) energy. The system 20 further includes a transformer 24 connected to an output of the RF generator 22. The RF generator 22 is connected to a primary winding 26 of the transformer.

The transformer has a secondary winding 28 which has a 1:1 ratio with respect to the primary winding 26 and, therefore, uses mutual inductance to achieve optimum energy transfer to the electrodes 16. The secondary winding 28 is centre tapped having a reference electrode 30 connected to a centre tap of the secondary winding 28. The centre tapped secondary winding 28 therefore provides two sources of electromagnetic energy with the energy of the sources being 180° out of phase.

The end electrode 16.1 of the ablation catheter 10 is connected to one of the sources provided by the secondary winding 28. The remaining electrodes 16 carried by the distal region 14 of the electrode sheath 12 are connected to the other source defined by the centre tapped secondary winding 28 via a switching arrangement 34. The switching arrangement 34 facilitates the switching of one of the remaining electrodes 16 into electrical contact with the other source. For example, in the illustrated embodiment, an electrode 16.2 carried by the distal region 14 of the electrode sheath 12 is being used and is, therefore, connected to the other source defined by the secondary winding 28 of the transformer 24 via the switching arrangement 34.

As described in the Applicant's International Patent Application No. PCT/AU2003/001421, ratios other than a 1:1 ratio between the primary winding 26 and the secondary winding 28 can be used for the transformer 24 with consequent variations in the current provided by the secondary winding 28.

The materials used in the transformer 14 are optimised to ensure maximum transfer of energy to the electrodes 16. Suitable materials for the transformer 24 include nickel-zinc or manganese-zinc ferrites for a core of the transformer 24, in particular, F8, F12, F14 ferrites. These materials are able to operate at the required frequencies and have the necessary high initial permeability and high saturation flux. The dimensions of the core of the transformer 24, the number of turns of the windings 26 and 28 and the diameter of the wire used for the windings are selected so that the transformer 14 has low insertion losses to ensure efficient transfer of energy.

In use, the catheter 10 is inserted into the vascular system of a patient's body and is steered to the site. Typically, an introducer (not shown) is used to introduce the catheter 10 into a femoral vein of the patient and the introducer is used to deliver the distal region 14 of the catheter to the site in an atrium of the heart of the patient. At the site, the distal region 14 of the catheter 10 is urged through the distal end of the introducer to enable the distal region 14 of the electrode sheath 12 of the catheter 10 to adopt the required shaped. To effect treatment of arrhythmias in a patient's heart, the distal region 14 of the electrode sheath 12 of the catheter 10 is urged against tissue at the site. The flexibility of the distal region 14 of the electrode sheath 12 helps to facilitate electrode-tissue contact at the site.

As described above, the electrode 16.1 is connected to one of the sources provided by the secondary winding 28 of the transformer 24. Depending on the shape of lesion desired to be formed, one of the other electrodes 16 is selected. In this example, electrode 16.2 is selected by appropriate manipulation of the switching arrangement 34.

The generator 22 is energised to provide 180° out of phase electromagnetic energy to the electrodes 16.1 and 16.2 via the secondary winding 28. The energy is supplied until overlapping zones 36 and 38 are formed as shown in FIG. 1 of the drawings to form a lesion extending between the electrodes 16.1 and 16.2. If the lesion comprising the overlapping zones 36 and 38 cures the arrhythmia, the procedure is complete. However, if the shape of the lesion is inadequate to cure the arrhythmia, a further electrode 16 of the remaining electrodes is selected. For example, it may be necessary to ablate tissue underlying electrode 16.3 and, therefore, electrode 16.3 is energised by appropriate manipulation of the switching arrangement 34. The RF energy is thus applied to the electrodes 16.1 and 16.3 to form further overlapping zones between electrodes 16.1 and 16.3 and, possibly, between electrodes 16.2 and 16.3 as well to increase the size of the lesion.

Thus, it will be appreciated that, by appropriate manipulation of the switching arrangement 34 appropriate pairs of electrodes 16 can be selected to obtain the desired shape of lesion.

Thus, with this arrangement, lesions of the required shape, depth and size can be formed by appropriate selection of the electrodes without having to manoeuvre the distal region 14 of the electrode sheath 12. This considerably facilitates the task of the clinician performing the procedure and allows for more accurate lesion formation.

It is therefore an advantage of the invention that a catheter 10 is provided which is less cumbersome and easier to operate for a clinician. Less manoeuvring of the catheter is required.

It is a further advantage of the invention that the stylet or steering shaft can be removed from the lumen of the electrode sheath 12 and replaced by a steering shaft having a different shape to form a differently shaped distal region 14 of the catheter 10 so as to provide different shapes and configurations of lesions.

Further, the use of at least two electrodes 16 is of benefit in creating overlapping lesions such as used in “Maze-like” procedures with the further advantage that, due to the use of electrodes on spaced arms of the distal region 14 of the electrode sheath 12, wider or “non-linear” lesions can more easily be formed.

It will be appreciated by persons skilled in the art that numerous variations and/or modifications may be made to the invention as shown in the specific embodiments without departing from the spirit or scope of the invention as broadly described. The present embodiments are, therefore, to be considered in all respects as illustrative and not restrictive.

1. A heat treatment catheter for use in heating a biological site in a patient's body, the heat treatment catheter including:
an elongate electrode-carrying element;
a plurality of heating electrodes arranged at spaced intervals at a distal region of the electrode-carrying element; and
the distal region of the electrode-carrying element being formed into a predetermined, non-rectilinear shape so that, in use, when any two of the electrodes are energised with heat energy, overlapping heat treated zones are created by the energised electrodes to form a heat treated region extending between the two energised electrodes.

2. The catheter of claim 1 in which the predetermined shape of the electrode-carrying element is a coiled or spiral shape.

3. The catheter of claim 2 in which the coiled or spiral shape of the distal region of the electrode-carrying element lies in a plane substantially transverse to a longitudinal axis of a remainder of the electrode-carrying element.

4. The catheter of claim 2 in which the electrode-carrying element is sufficiently flexible so that, at least when the spiral shape distal region of the electrode-carrying element is urged against the site to be treated, the spiral shape is able to mould to the shape of the site and flex to accommodate surface irregularities at the site.

5. The catheter of claim 1 in which the electrode-carrying element is steerable, the electrode-carrying element having a lumen in which a steering mechanism is received.

6. The catheter of claim 1 in which an electrode at a distal end of the electrode-carrying element and any one other electrode are employed, in use.

7. The catheter of claim 6 in which the electrodes to be energised simultaneously are energised by out-of-phase electrical sources.

8. A system for heating a biological site in a patient's body, the system including:
an electrode-carrying element having a plurality of electrodes arranged at spaced intervals at a distal region, the distal region of the electrode-carrying element being arranged in a predetermined non-rectilinear shape; and
a source of electromagnetic energy connectable to the electrode-carrying element to energise at least two of the electrodes simultaneously, with the electromagnetic energy associated with one electrode being out of phase with the electromagnetic energy associated with any one other, simultaneously energised electrode.

9. The system of claim 8 in which the source of electromagnetic energy is a transformer having a primary winding and a secondary winding, the secondary winding having at least one tap to provide a ground reference and at least two sources of heat energy.

10. The system of claim 9 in which a secondary winding of the transformer has a 1:1 ratio with respect to a primary winding of the transformer.

11. The system of claim 9 in which the electrode at a distal end of the electrode-carrying element is always connected to one of the sources of heat energy.

12. The system of claim 11 which includes a switching arrangement, connected to the other of the sources of heat energy, for switching any one of the remaining electrodes, at any one time, into electrical contact with that other source of heat energy.

13. The system of claim 9 in which the primary winding of the transformer is connectable to an energy generator for supplying the heat energy to the primary winding of the transformer.

14. The system of claim 9 in which the secondary windings of the transformer supply energy to the electrodes connected to the secondary windings with the energy supplied to one of the electrodes being 180° out of phase with the energy supplied to the other connected electrode.

15. A method of heating a site in a patient's body, the method including:
positioning a distal region of an electrode-carrying element relative to the site, the distal region of the electrode-carrying element having a plurality of electrodes arranged at spaced intervals and the distal region of the electrode-carrying element being non-rectilinear in shape;
supplying energy to at least two of the electrodes simultaneously with the energy supplied to each of the electrodes being out of phase; and
maintaining the energy supply to the energised electrodes until overlapping heat treated zones are formed by the electrodes to form a heat treated region extending between the two energised electrodes.

16. The method of claim 15 in which the distal region of the electrode-carrying element is in the form of a spiral or coiled shape so that the electrodes lie in spaced relationship on adjacent, but spaced, turns of the spiral shaped end of the electrode-carrying element and in which the method includes forming the overlapping zones by using the electrode at the distal end of the electrode-carrying element and at least one other electrode to form a desired shape of region in a desired position at the site.
